(19) United States
(12) Patent Application Publication
(10) Pub. No.: US 2026/0325079 A1
(43) Pub. Date: Oct. 22, 2026

(54) COLLECTING DATA AND ASSESSING DATA COLLECTION IN REAL-TIME

(71) Applicant: International Business Machines Corporation, Armonk, NY (US)

(72) Inventors: Jenna REINEN, Rye Brook, NY (US); Carla Paola AGURTO RIOS, Ossining, NY (US); Raquel NOREL, Bronx, NY (US); Chen WANG, Chappaqua, NY (US)

(21) Appl. No.: 19/183,590

(22) Filed: Apr. 18, 2025

(51) Int. Cl. A61B 5/00; A61B 5/11

(52) U.S. Cl. CPC A61B 5/7221; A61B 5/11; A61B 5/4088; A61B 5/4806

(57) ABSTRACT

In some implementations, a computer system may receive data collection requirements, and may determine data collection settings based on the data collection requirements. The computer system may receive data associated with a user of a user device based on the data collection settings, and may perform feature extraction to extract features from the data. The computer system may determine whether the data collection requirements are satisfied by the data based on the features. The computer system may selectively adjust the data collection settings based on determining that the data collection requirements are not satisfied and to generate adjusted data collection settings, or may terminate collection of the data associated with the user based on determining that the data collection requirements are satisfied.

### BACKGROUND

This disclosure relates to collecting data, and more specifically, to an intelligent adaptive system for collecting data and assessing data collection in real-time.

### SUMMARY

Some implementations described herein relate to a method. The method may include receiving data collection requirements, and determining data collection settings based on the data collection requirements. The method may include receiving data associated with a user of a user device based on the data collection settings, and performing feature extraction to extract features from the data. The method may include determining whether the data collection requirements are satisfied by the data based on the features. The method may include selectively adjusting the data collection settings based on determining that the data collection requirements are not satisfied and to generate adjusted data collection settings, or terminating collection of the data associated with the user based on determining that the data collection requirements are satisfied.

Some implementations described herein relate to a computer system. The computer system may include a processor set, one or more computer-readable storage media, and program instructions stored on the one or more computer-readable storage media to cause the processor set to perform operations. The operations may include receiving data collection requirements, and determining data collection settings based on the data collection requirements. The operations may include generating instructions based on the data collection settings, and providing the instructions to a user of a user device. The operations may include receiving data associated with the user of the user device based on the instructions, performing feature extraction to extract features from the data, and determining whether the data collection requirements are satisfied by the data based on the features. The operations may include selectively adjusting the data collection settings based on determining that the data collection requirements are not satisfied and to generate adjusted data collection settings, or terminating collection of the data associated with the user based on determining that the data collection requirements are satisfied.

Some implementations described herein relate to a computer program product. The computer program product may include one or more computer-readable storage media and program instructions stored on the one or more computer-readable storage media to perform operations. The operations may include receiving data collection requirements, and determining data collection settings based on the data collection requirements. The operations may include receiving data associated with a user of a user device based on the data collection settings, performing feature extraction to extract features from the data, and determining whether the data collection requirements are satisfied by the data based on the features. The operations may include selectively adjusting the data collection settings based on determining that the data collection requirements are not satisfied and utilizing the data collection settings, as adjusted, to collect additional data associated with the user, or terminating collection of the data associated with the user based on determining that the data collection requirements are satisfied.

### BRIEF DESCRIPTION OF THE DRAWINGS

FIG. 1 is a diagram of an example computing environment for collecting data and assessing a data collection in real-time as described herein.

FIGS. 2A-2E are diagrams of an example associated with collecting data and assessing data collection in real-time as described herein.

FIG. 3 is a diagram of example components of one or more devices of FIGS. 1 and 2A-2E.

FIG. 4 is a flowchart of an example process associated with collecting data and assessing data collection in real-time.

### DETAILED DESCRIPTION

The following detailed description of example implementations refers to the accompanying drawings. The same reference numbers in different drawings may identify the same or similar elements.

Clinical and research settings increasingly employ sensor-based technologies for continuous patient monitoring. Such monitoring generates a vast amount of data associated with physiological signals, movement patterns, speech characteristics, and/or the like. The sheer volume of this data poses significant challenges in terms of data management and analysis. Furthermore, prolonged monitoring periods can burden patients, impacting their compliance, stress levels, and overall experience. Traditional data collection methods often involve collecting large amounts of data without real-time assessment of data quality or relevance. This can result in unnecessary data being collected, increasing the burden on patients and the resources required for data analysis and storage. Moreover, a lack of real-time data quality control can lead to low-quality or irrelevant data being collected, which may not provide meaningful insights or may even lead to incorrect conclusions. This can undermine the validity and reliability of research findings, ultimately affecting the development of effective treatments and interventions. In addition, data collection is often not tailored to specific needs of individual patients or research studies, which may cause the data collection to continue beyond a point at which sufficient information has been gathered. Thus, traditional data collection methods consume computing resources (e.g., processing resources, memory resources, communication resources, and/or the like), networking resources, and/or the like associated with collecting unnecessary, irrelevant, or low-quality data, generating incorrect conclusions based on the unnecessary, irrelevant, or low-quality data, failing to provide effective treatment based on the incorrect conclusions, unnecessarily collecting data after sufficient data has been captured, and/or the like.

Some implementations described herein provide a computer system for collecting data and assessing data collection in real-time. For example, the computer system may receive data collection requirements, and may determine data collection settings based on the data collection requirements. The computer system may receive data associated with a user of a user device based on the data collection settings, and may perform feature extraction to extract features from the data. The computer system may determine whether the data collection requirements are satisfied by the data based on the features. The computer system may selectively adjust the data collection settings based on determining that the data collection requirements are not satisfied and to generate adjusted data collection settings, or may terminate collection of the data associated with the user based on determining that the data collection requirements are satisfied.

In this way, the computer system collects data and assesses data collection in real-time. For example, the computer system may provide an adaptive and efficient data collection process that minimizes data redundancy, reduces data transmission overhead, and conserves resources. By continuously assessing data quality and relevance in real-time, the computer system may optimize data collection by reducing a volume of data required for analysis, thereby decreasing computational complexity, and facilitating faster data processing and analysis. Additionally, the computer system may utilize feature extraction to extract features from the collected data, and may determine whether data collection requirements are satisfied by the data based on the features. When the data collection requirements are satisfied, the computer system may terminate the data collection, conserving resources and reducing stress on users providing the data. When the data collection requirements are not satisfied, the computer system may adjust data collection settings and may utilize the adjusted data collection settings to collect additional data. Thus, the computer system conserves computing resources, networking resources, and/or the like that would otherwise have been consumed by collecting unnecessary, irrelevant, or low-quality data, generating incorrect conclusions based on the unnecessary, irrelevant, or low-quality data, failing to provide effective treatment based on incorrect conclusions, unnecessarily collecting data after sufficient data has been captured, and/or the like.

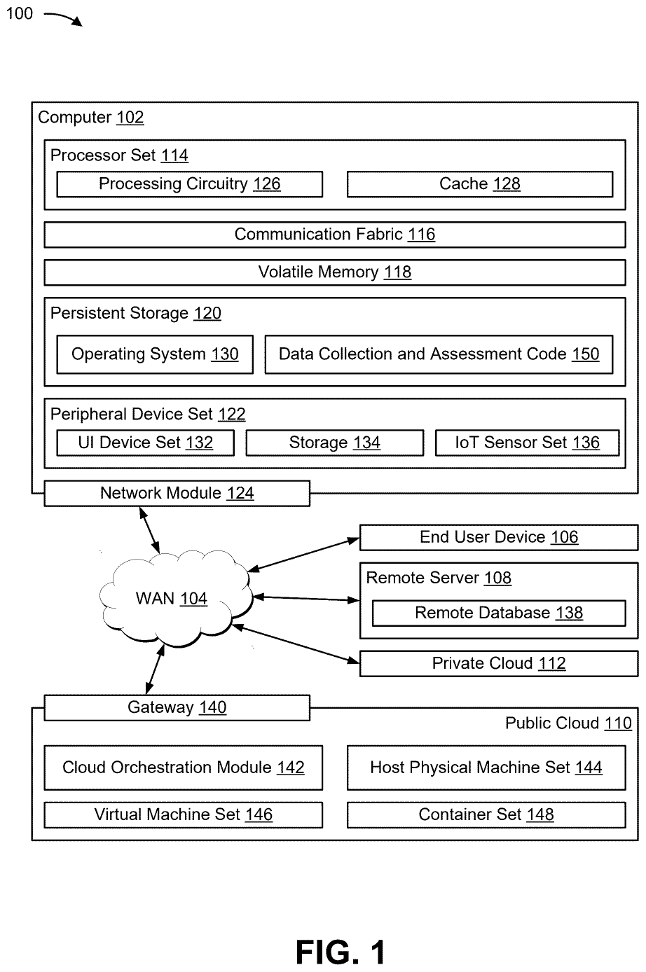

FIG. 1 is a diagram of an example computing environment 100 for collecting data and assessing data collection in real-time, as described herein.

Computing environment 100 contains an example of an environment for the execution of at least some of the computer code involved in performing the inventive methods, such as data collection and assessment code 150. In addition to data collection and assessment code 150, computing environment 100 includes, for example, a computer 102, a wide area network (WAN) 104, an end user device (EUD) 106, a remote server 108, a public cloud 110, and a private cloud 112. In this embodiment, computer 102 includes a processor set 114 (including processing circuitry 126 and a cache 128), a communication fabric 116, a volatile memory 118, persistent storage 120 (including an operating system 130 and data collection and assessment code 150, as identified above), a peripheral device set 122 (including a user interface (UI) device set 132, storage 134, and an Internet of Things (IoT) sensor set 136), and a network module 124. Remote server 108 includes a remote database 138. Public cloud 110 includes a gateway 140, a cloud orchestration module 142, a host physical machine set 144, a virtual machine set 146, and a container set 148.

Computer 102 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network, or querying a database, such as remote database 138. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 102, to keep the presentation as simple as possible. Computer 102 may be located in a cloud, even though it is not shown in a cloud in FIG. 1. On the other hand, computer 102 is not required to be in a cloud except to any extent as may be affirmatively indicated.

Processor set 114 includes one, or more, computer processors of any type now known or to be developed in the future. Processing circuitry 126 may be distributed over multiple packages (for example, multiple, coordinated integrated circuit chips). Processing circuitry 126 may implement multiple processor threads and/or multiple processor cores. Cache 128 is memory that is located in the processor chip package(s) and is typically used for data or code that should be available for rapid access by the threads or cores running on processor set 114. Cache memories are typically organized into multiple levels depending upon relative proximity to the processing circuitry. Alternatively, some, or all, of the cache for the processor set may be located “off chip.” In some computing environments, processor set 114 may be designed for working with qubits and performing quantum computing.

Computer-readable program instructions are typically loaded onto computer 102 to cause a series of operational steps to be performed by processor set 114 of computer 102 and thereby effect a computer-implemented method, such that the instructions thus executed will instantiate the methods specified in flowcharts and/or narrative descriptions of computer-implemented methods included in this document (collectively referred to as “the inventive methods”). These computer-readable program instructions are stored in various types of computer-readable storage media, such as cache 128 and the other storage media discussed below. The program instructions, and associated data, are accessed by processor set 114 to control and direct performance of the inventive methods. In computing environment 100, at least some of the instructions for performing the inventive methods may be stored in data collection and assessment code 150 in persistent storage 120.

Communication fabric 116 is the signal conduction path that allows the various components of computer 102 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

Volatile memory 118 is any type of volatile memory now known or to be developed in the future. Examples include dynamic type random access memory (RAM) or static type RAM. Typically, volatile memory 118 is characterized by random access, but this is not required unless affirmatively indicated. In computer 102, the volatile memory 118 is located in a single package and is internal to computer 102, but, alternatively or additionally, the volatile memory may be distributed over multiple packages and/or located externally with respect to computer 102.

Persistent storage 120 is any form of non-volatile storage for computers that is now known or to be developed in the future. The non-volatility of this storage means that the stored data is maintained regardless of whether power is being supplied to computer 102 and/or directly to persistent storage 120. Persistent storage 120 may be a read only memory (ROM), but typically at least a portion of the persistent storage allows writing of data, deletion of data, and re-writing of data. Some familiar forms of persistent storage include magnetic disks and solid-state storage devices. Operating system 130 may take any of several forms, such as various known proprietary operating systems or open source Portable Operating System Interface-type operating systems that employ a kernel.

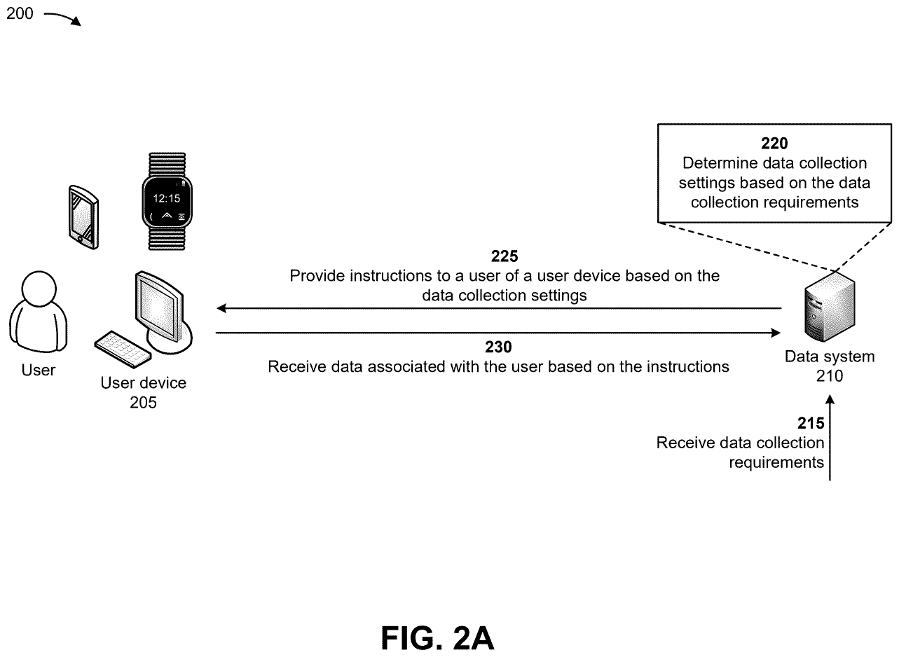

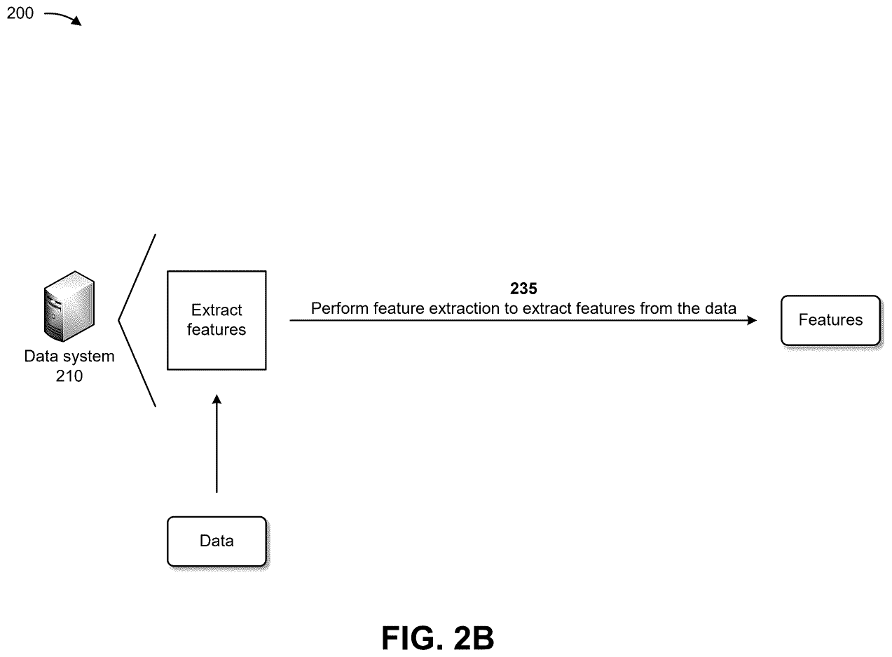

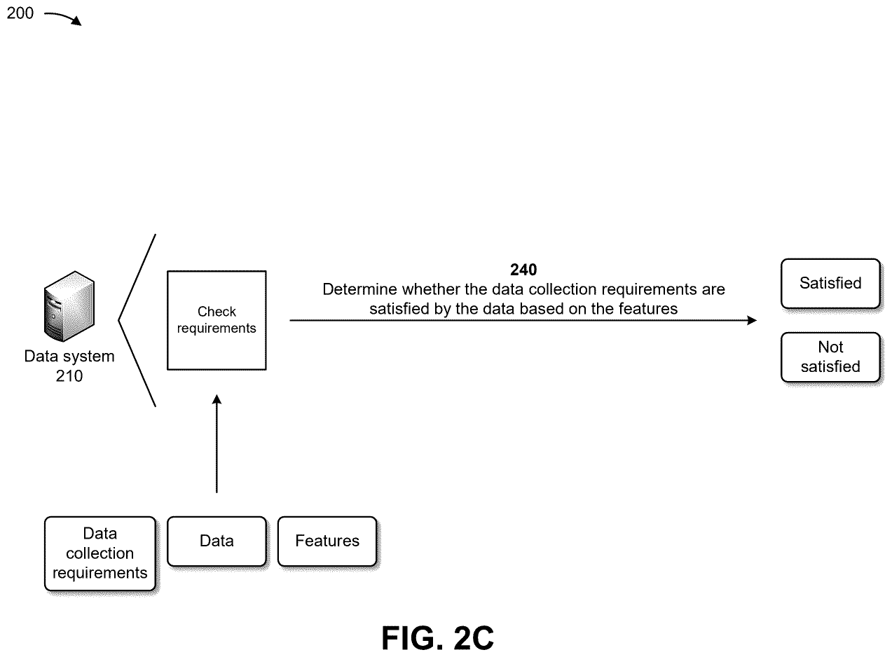

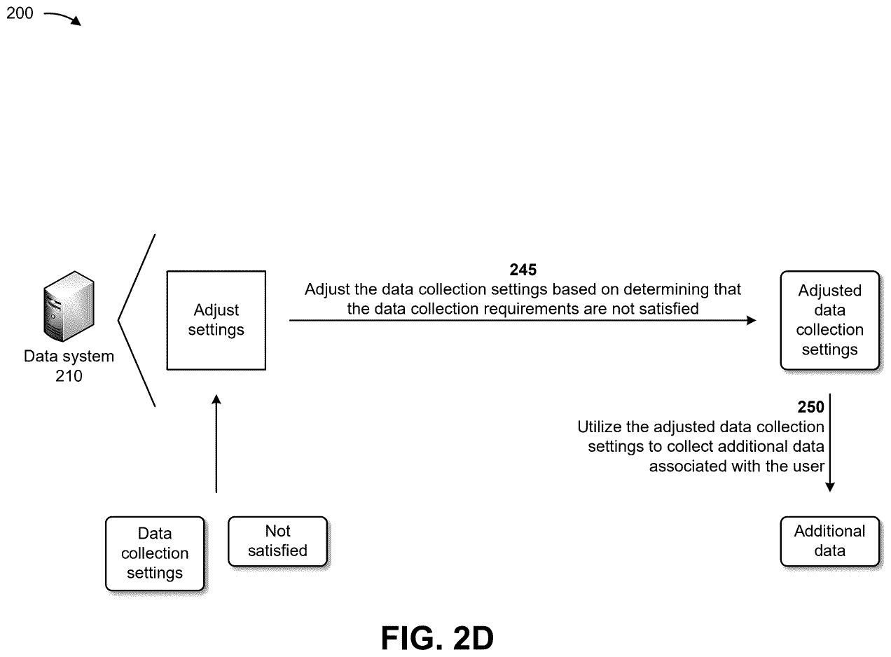

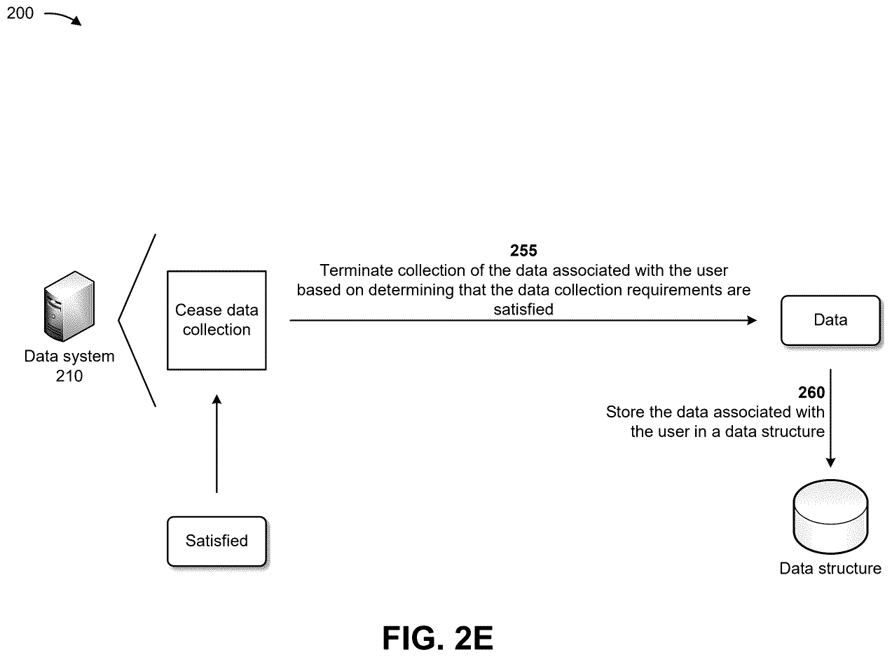

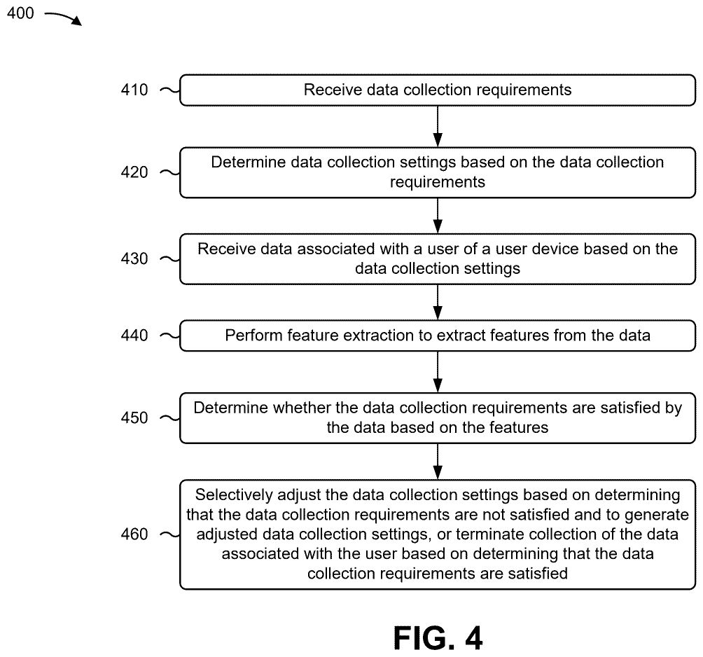

The code included in the data collection and assessment code 150 typically includes at least some of the computer code involved in performing one or more operations described herein, such as the operations of implementation 200 in FIGS. 2A-2E and the process described in FIG. 4.

Peripheral device set 122 includes the set of peripheral devices of computer 102. Data communication connections between the peripheral devices and the other components of computer 102 may be implemented in various ways, such as Bluetooth® connections, Near-Field Communication (NFC) connections, connections made by cables (such as universal serial bus (USB) type cables), insertion-type connections (for example, secure digital (SD) card), connections made through local area communication networks and/or connections made through wide area networks such as the internet. In various embodiments, UI device set 132 may include components such as a display screen, speaker, microphone, wearable devices (such as goggles and smart watches), keyboard, mouse, printer, touchpad, game controllers, and/or haptic devices. Storage 134 is external storage, such as an external hard drive, or insertable storage, such as an SD card. Storage 134 may be persistent and/or volatile. In some embodiments, storage 134 may take the form of a quantum computing storage device for storing data in the form of qubits. In embodiments where computer 102 is required to have a large amount of storage (for example, where computer 102 locally stores and manages a large database), this storage may be provided by peripheral storage devices designed for storing very large amounts of data, such as a storage area network (SAN) that is shared by multiple, geographically distributed computers. IoT sensor set 136 is made up of sensors that can be used in Internet of Things applications. For example, one sensor may be a thermometer and another sensor may be a motion detector.

Network module 124 is the collection of computer software, hardware, and firmware that allows computer 102 to communicate with other computers through WAN 104. Network module 124 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 124 are performed on the same physical hardware device. In other embodiments (for example, embodiments that utilize software-defined networking (SDN)), the control functions and the forwarding functions of network module 124 are performed on physically separate devices, such that the control functions manage several different network hardware devices. Computer-readable program instructions for performing the inventive methods can typically be downloaded to computer 102 from an external computer or external storage device through a network adapter card or network interface included in network module 124.

WAN 104 is any wide area network (for example, the internet) capable of communicating computer data over non-local distances by any technology for communicating computer data, now known or to be developed in the future. In some embodiments, the WAN 104 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and/or edge servers.

EUD 106 is any computer system that is used and controlled by an end user (for example, a customer of an enterprise that operates computer 102), and may take any of the forms discussed above in connection with computer 102. EUD 106 typically receives helpful and useful data from the operations of computer 102. For example, in a hypothetical case where computer 102 is designed to provide a recommendation to an end user, this recommendation would typically be communicated from network module 124 of computer 102 through WAN 104 to EUD 106. In this way, EUD 106 can display, or otherwise present, the recommendation to an end user. In some embodiments, EUD 106 may be a client device, such as thin client, heavy client, mainframe computer, desktop computer and so on.

Remote server 108 is any computer system that serves at least some data and/or functionality to computer 102. Remote server 108 may be controlled and used by the same entity that operates computer 102. Remote server 108 represents the machine(s) that collect and store helpful and useful data for use by other computers, such as computer 102. For example, in a hypothetical case where computer 102 is designed and programmed to provide a recommendation based on historical data, this historical data may be provided to computer 102 from remote database 138 of remote server 108.

Public cloud 110 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 110 is performed by the computer hardware and/or software of cloud orchestration module 142. The computing resources provided by public cloud 110 are typically implemented by virtual computing environments that run on various computers making up the computers of host physical machine set 144, which is the universe of physical computers in and/or available to public cloud 110. The virtual computing environments (VCEs) typically take the form of virtual machines from virtual machine set 146 and/or containers from container set 148. These VCEs may be stored as images and may be transferred among and between the various physical machine hosts, either as images or after instantiation of the VCE. Cloud orchestration module 142 manages the transfer and storage of images, deploys new instantiations of VCEs, and manages active instantiations of VCE deployments. Gateway 140 is the collection of computer software, hardware, and firmware that allows public cloud 110 to communicate through WAN 104.

Some further explanation of VCEs will now be provided. VCEs can be stored as “images.” A new active instance of a VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 112 is similar to public cloud 110, except that the computing resources are only available for use by a single enterprise. While private cloud 112 is depicted as being in communication with WAN 104, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this example, public cloud 110 and private cloud 112 are both part of a larger hybrid cloud.

Cloud computing services and/or microservices (not separately shown in FIG. 1): private and public clouds 110 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider's systems, and back. In some embodiments, cloud services may be configured and orchestrated according to an “as a service” technology paradigm where content is being presented to an internal or external customer in the form of a cloud computing service. As-a-service offerings typically provide endpoints with which various customers interface. These endpoints are typically based on a set of application programming interfaces (APIs). One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with such tasks. Another category is Software-as-a-Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

The number and arrangement of devices and networks shown in FIG. 1 are provided as an example. In practice, there may be additional devices and/or networks, fewer devices and/or networks, different devices and/or networks, or differently arranged devices and/or networks than those shown in FIG. 1. Furthermore, two or more devices shown in FIG. 1 may be implemented within a single device, or a single device shown in FIG. 1 may be implemented as multiple, distributed devices. Additionally, or alternatively, a set of devices (e.g., one or more devices) of the computing environment 100 may perform one or more functions described as being performed by another set of devices of the computing environment 100.

FIGS. 2A-2E are diagrams of an example 200 associated with collecting data and assessing a data collection in real-time as described herein. As shown in FIGS. 2A-2E, the example 200 includes a user device 205 associated with a user (e.g., an end user) and a data system 210. The user device 205 may correspond to the computer 102, the end user device 106, a wearable device (e.g., a smartwatch, a fitness tracker, and/or the like), a sensor (e.g., an accelerometer), medical equipment (e.g., a heartbeat monitor or a blood pressure monitor), and/or the like. In some implementations, the user may be associated with multiple user devices 205 (e.g., a sensor, a smartphone, and/or the like). The data system 210 may correspond to the computer 102, the end user device 106, the remote server 108, a system provided by the public cloud 110, and/or a system provided by the private cloud 112. Further details of the user device 205 and the data system 210 are provided elsewhere herein.

As shown in FIG. 2A, and by reference number 215, the data system 210 may receive data collection requirements. For example, the data system 210 may receive data collection requirements that specify particular data types, quality criteria, and collection settings tailored for a specific study, experiment, or application. The data collection requirements may include specifications or criteria that define the types of data to be collected, the quality thresholds for the data, and the parameters for the data collection process. The data collection requirements may tailored to specific needs of a study, an experiment, or an application.

In some implementations, the data system 210 may generate a user interface that requests input of the data collection requirements, and may provide the user interface for display to an operator (e.g., a scientist, a researcher, and/or the like) of the data system 210 (e.g., via a user device associated with the operator). The operator may utilize the user interface to input the data collection requirements, and the data system 210 may receive the data collection requirements via the input to the user interface. In some implementations, the data collection system 210 may preconfigure the data collection requirements based on needs of the study, the experiment, or the application. In some implementations, the data system 210 may receive the data collection requirements from various sources. For example, the data collection requirements may be input by a researcher through the user interface, may be automatically generated based on predefined study protocols, may be received via an API of a third-party system or application, and/or the like. Thus, the data system 210 may ensure seamless integration with existing data management workflows, and may efficiently incorporate advanced study protocols and industry standards.

As further shown in FIG. 2A, and by reference number 220, the data system 210 may determine data collection settings based on the data collection requirements. For example, the data system 210 may analyze the received data collection requirements to configure appropriate data collection settings to be implemented by the user device 205. The data collection settings may include settings that define sensor types (e.g., a smartphone microphone, an accelerometer, a heartrate monitor, and/or the like) utilized to capture data, data capture intervals (e.g., in seconds, minutes, and/or the like), quality thresholds for the captured data, and/or the like. The data collection settings may ensure that the data collection by the data system 210 is aligned with specific goals and parameters of a study, an experiment, or an application. In some implementations, the data system 210 may utilize one or more machine learning models to determine the data collection settings. The one or more machine learning models may optimize the data collection settings based on historical data and the specific objectives of the study, the experiment, or the application. The one or more machine learning models may identify patterns and may recommend modifications to the data collection settings to improve data accuracy and relevance. Additionally, or alternatively, the data system 210 may dynamically determine the data collection settings, and may adjust the data collection settings in real-time based on initial data quality assessments and feedback loops. This dynamic adjustment may enable continuous improvement of data collection processes and may provide for immediate correction of any discrepancies.

As further shown in FIG. 2A, and by reference number 225, the data system 210 may provide instructions to a user of the user device 205 based on the data collection settings. For example, the data system 210 may generate instructions that implement the data collection settings and that guide the user on how to setup and operate the user device 205 for optimal data collection. The instructions may include prompts to start data capture, guidelines for proper sensor use, notifications about data collection status (e.g., a progress bar), and/or the like. The data system 210 may provide the instructions to the user device 205, and the user device 205 may implement the instructions so that the user device 205 may capture data associated with the user (e.g., movement of the user, a voice of the user, a heartbeat of the user, snoring by the user, and/or the like). In some implementations, the user device 205 may provide the instructions to the user in the form of visual aids, such as diagrams and videos, to ensure proper setup and operation of the user device 205 for data collection. The visual aids may enhance user understanding and compliance with the data collection settings. Additionally, or alternatively, the data system 210 may provide the instructions through an interactive chatbot on the user device 205, offering real-time assistance and troubleshooting tips to the user during the data collection process. The interactive chatbot may respond to user queries and may provide step-by-step guidance, improving the user experience and data reliability.

As further shown in FIG. 2A, and by reference number 230, the data system 210 may receive data associated with the user based on the instructions. For example, after the user has followed the instructions provided by the data system 210, the user device 205 may start collecting data associated with the user, such as physiological signals, movement patterns, speech characteristics, snoring of the user, and/or the like. The user device 205 may provide collected data associated with the user to the data system 210 in real-time. The data system 210 may receive the data in real-time for further processing and analysis. Real-time may include an ability to process and analyze data almost instantaneously as the data is received. Real-time data processing may ensure timely interventions and adjustments to optimize data collection by the data system 210. In some implementations, the data system 210 may receive the data associated with the user not only from the user device 205 but also from other connected devices and sensors. The data system 210 may aggregate the data received from multiple sources to enhance the analysis of the data collection. A multi-source data collection may provide a comprehensive view of the user's activities and conditions. Additionally, or alternatively, the data system 210 may receive the data associated with the user via a secure cloud platform. The secure cloud platform may ensure data integrity and accessibility for remote monitoring and analysis, and may provide scalable and secure data storage and access. Additionally, or alternatively, the data system 210 may receive and store the data associated with the user in a decentralized manner. For example, the data system 210 may utilize blockchain technology to ensure data security and traceability throughout the data collection process. Blockchain technology may provide tamper-proof records and may enhance data integrity for sensitive user data.

As shown in FIG. 2B, and by reference number 235, the data system 210 may perform feature extraction to extract features from the data. For example, the data system 210 may analyze the data associated with the user, and may identify and extract features relevant to the data collection requirements based on analyzing the data. Feature extraction may include a process of analyzing raw data to identify and extract relevant characteristics or patterns (e.g., features) that can be used to evaluate the data collection requirements. The feature extraction process may utilize various analytical models and techniques. The features may include parameters such as physiological signals, movement patterns, speech characteristics, and/or the like, depending on the nature of the data associated with the user. The feature extraction process may include applying various models and techniques to the data associated with the user to derive meaningful metrics and patterns. In some implementations, the data system 210 may utilize one or more machine learning models, natural language processing models, or other analytical tools to perform the feature extraction on the data associated with the user. The extracted features may enable the data system 210 to effectively evaluate the quality and relevance of the collected data in real-time, and to provide precise assessment and decision-making for adaptive and efficient data collection.

In some implementations, the features may include parameters such as accelerometer variability, linguistic content, vocabulary diversity, acoustic features, syntactic complexity, frequency and intensity of snoring or coughing episodes, and/or the like. For example, the data system 210 may analyze accelerometer data to identify variability patterns, may assess linguistic content to identify diversity and coherence, and may evaluate acoustic features such as speech fluency and intensity of snoring or coughing episodes. In some implementations, the feature extraction process may include evaluating the data within short, predefined time windows. For example, the data system 210 may assess accelerometer data in ten-minute intervals and linguistic content in ten-second intervals to ensure timely and accurate feature extraction.

In some implementations, the feature extraction process may include using specific pre-trained machine learning models designed for various types of data. For example, when dealing with physiological signals, a convolutional neural network (CNN) pre-trained on a large dataset of physiological signals may be used to extract relevant features such as heart rate variability, signal amplitude, and signal frequency. The training process for such a model may include supervised learning with labeled data, where the model learns to recognize patterns associated with different physiological states. For linguistic content analysis, a transformer-based natural language processing model, such as a bidirectional encoder representations from transformers (BERT) model, may be utilized for feature extraction. This model may be fine-tuned on a dataset of speech recordings to recognize features such as vocabulary diversity, syntactic complexity, and sentiment. The fine-tuning process may include adjusting the model's weights based on a smaller, task-specific dataset to improve its performance on the target task.

As shown by FIG. 2C, and reference number 240, the data system 210 may determine whether the data collection requirements are satisfied by the data based on the features. For example, the data system 210 may analyze the extracted features to evaluate alignment of the features with the data collection requirements. The data system 210 may assess a quality and a relevance of the collected data by comparing the extracted features to criteria specified in the data collection requirements. In some implementations, the data system 210 may continuously capture and analyze data streams, may immediately flag any deviations from the data collection requirements, and may adjust data collection processes accordingly to maintain the integrity of the collected data. In some implementations, the data system 210 may determine that the data collection requirements are satisfied by the data based on the features (e.g., and the data associated with the user). Alternatively, the data system 210 may determine that the data collection requirements are not satisfied by the data based on the features.

The data system 210 may determine whether the data collection requirements are satisfied by applying various models to the extracted features to ensure that the collected data satisfies standards for accuracy, completeness, and significance. For example, the data system 210 may utilize machine learning models, natural language processing models, deep learning models, statistical models, large language models, and/or the like to determine whether the data collection requirements are satisfied by the data based on the features. A machine learning model may include a computational model that uses algorithms to learn from data and make predictions or decisions based on that data. A machine learning model may include a supervised learning model, an unsupervised learning model, or a reinforcement learning model. A natural language processing model may include a type of computational model designed to analyze and understand human language. A natural language processing model may be used to process and analyze text data, extract relevant features, and assess whether the data collection requirements are satisfied. A large language model may include a type of machine learning model specifically designed to process and generate human-like text by analyzing large datasets of text. An example of a large language model may include a transformer-based model.

The machine learning models may include supervised learning models (e.g., logistic regression, decision trees, and random forests), unsupervised learning models (e.g., clustering and dimensionality reduction), or reinforcement learning models. The machine learning models may learn patterns and relationships between the features and the data collection requirements, enabling the data system 210 to make informed decisions. When dealing with text or speech data, natural language processing models, such as language models, sentiment analysis models, or topic modeling techniques may extract relevant features and assess whether the features satisfy the data collection requirements. The deep learning models may include neural networks, convolutional neural networks (CNNs), or recurrent neural networks (RNNs). The deep learning models may analyze complex data types, such as images, audio, or time-series data, and determine whether the features satisfy the data collection requirements based on the analysis. The statistical models, such as hypothesis testing, confidence intervals, or regression analysis, may also be employed to evaluate the features and determine whether the features satisfy the data collection requirements. The large language models, such as transformer-based models, may be utilized to process and analyze large amounts of text data, extract relevant features, and assess whether the features satisfy the data collection requirements. These models can be used individually or in combination to determine whether the data collection requirements are satisfied by the data based on the features. The choice of models may depend on a specific use case, the type of data being collected, and the complexity of the data collection requirements.

As shown in FIG. 2D, and by reference number 245, the data system 210 may adjust the data collection settings based on determining that the data collection requirements are not satisfied. For example, when the data system 210 determines that the features (e.g., and the data associated with the user) fail to satisfy the data collection requirements, the data system 210 may adjust the data collection settings to generate adjusted data collection settings that improve the quality or relevance of the collected data. The adjustments to the data collection settings may include changes to one or more sensor configurations, changes to data capture intervals, changes to quality thresholds, and/or the like. For example, the data system 210 may switch from an accelerometer to a gyroscope for capturing movement data, may increase a frequency of data capture for a more granular analysis, may lower a threshold for acceptable data quality, and/or the like, to include more diverse data points. In some implementations, the data system 210 may utilize one or more machine learning models to adjust the data collection settings based on historical data and specific objectives. For example, the data system 210 may utilize a machine learning model that analyzes past data collection performance and adjusts settings to enhance future data quality and relevance. This adaptive approach may ensure that the data collection process remains aligned with objectives and parameters of a study, an experiment, or an application, resulting in more accurate and meaningful data.

In some implementations, the data system 210 may utilize a reinforcement learning model, such as a Q-learning model, to dynamically adjust data collection settings. The model may continuously evaluate the quality of incoming data against the data collection requirements. If the data quality fails to satisfy the data collection requirements, the model may make incremental adjustments to the data collection settings, such as modifying sensor configurations or data capture intervals. These adjustments may be based on a reward function that prioritizes data quality and relevance. The reinforcement learning model may be trained in a simulated environment to optimize the adjustment strategy before being deployed in a real-world setting.

As further shown in FIG. 2D, and by reference number 250, the data system 210 may utilize the adjusted data collection settings to collect additional data associated with the user. For example, after adjusting the data collection settings, the data system 210 may generate updated instructions based on the adjusted data collection settings. The data system 210 may provide the updated instructions to the user device 205. The user device 205 may continue to collect data based on the updated instructions and the adjusted data collection settings, and may capture additional data that is more likely to meet the data collection requirements. This iterative process of data collection and adjustment may enable the data system 210 to dynamically respond to real-time assessments of data quality and relevance, thereby optimizing the overall data collection process and reducing unnecessary data capture. In some implementations, the data system 210 may adjust data collection settings in specific scenarios, such as monitoring accelerometer variability in physical activity, linguistic content analysis in cognitive assessments, and acoustic feature detection in sleep studies. For example, during a physical activity monitoring session, the data system 210 may adjust a data capture interval of the accelerometer to better capture periods of high variability in movement.

As shown by FIG. 2E, and reference number 255, the data system 210 may terminate collection of the data associated with the user based on determining that the data collection requirements are satisfied. For example, when the data system 210 determines that the features (e.g., and the data associated with the user) satisfy the data collection requirements, the data system 210 may terminate the collection of the data associated with the user. In some implementations, the data system 210 may terminate the collection of the data associated with the user by generating an instruction that instructs the user device 205 to cease providing the data associated with the user to the data system 210. For example, the data system 210 may provide the instruction to the user device 205, and the user device 205 may terminate collection and provision of the data to the data system 210. Alternatively, or additionally, the data system 210 may terminate the collection of the data associated with the user by preventing the user device 205 from providing the data to the data system 210, by discarding any additional data received from the user device 205, and/or the like. In this way, the data system 210 may avoid unnecessary data accumulation and optimize resource usage. By terminating further data collection, the data system 210 may ensure that only the necessary data is captured, which can help in managing storage capacity and processing efficiency. Furthermore, terminating further data collection may ensure that the user experience is not adversely affected by prolonged data collection periods. Additionally, or alternatively, the data system 210 may verify satisfaction of the data collection requirements prior to terminating the collection of the data associated with the user. Additionally, or alternatively, after terminating the collection of the data associated with the user, the data system 210 may switch to a low-power mode or a standby state and/or may cause the user device to switch to a low-power mode or a standby state, further conserving energy and computational resources.

As further shown in FIG. 2E, and by reference number 260, the data system 210 may store the data associated with the user in a data structure. For example, the data system 210 may organize and store the data (e.g., in a structured format) in a data structure (e.g., a database, a table, a list, and/or the like) associated with the data system 210. In some implementations, the data system 210 may organize the collected data in the data structure in a manner that facilitates efficient retrieval and analysis for subsequent studies or applications. This organization may include tagging data with relevant metadata for easier access. Additionally, or alternatively, the data system 210 may store the data in a hierarchical or relational database format, enabling structured queries and efficient data management. This may provide for complex queries to be performed on the stored data to extract meaningful insights. Additionally, or alternatively, the data system 210 may upload the collected data to a cloud storage service for centralized analysis and long-term storage. This may provide scalable storage options and may support collaborative analysis by multiple stakeholders. Additionally, or alternatively, the data system 210 may maintain a log or audit trail of the data collection process, and may document times at which data collection was initiated and terminated for future reference and verification. The log may be crucial for ensuring data integrity and compliance with data governance standards. Additionally, or alternatively, the data system 210 may implement encryption or other security measures to ensure that the stored data remains secure and compliant with privacy regulations and that sensitive user information is protected against unauthorized access. Additionally, or alternatively, the data system 210 may periodically back up the stored data to prevent data loss and ensure data integrity for future use. Regular backups may mitigate risks associated with data corruption or hardware failures.

In some implementations, the data system 210 may be utilized with accelerometer data for monitoring a variability in physical activity. For example, a clinical trial may aim to assess an impact of a new medication on physical activity levels of patients with chronic obstructive pulmonary disease (COPD). The participants may wear accelerometers that record their movements continuously over a period of several weeks. The data system 210 may be integrated with the accelerometers to monitor variability in physical activity data. During the trial, the data system 210 may evaluate the accelerometer data within short time windows (e.g., ten-minute intervals) to assess consistency and variability of the participants' movements. The data system 210 may determine whether the patient has moved enough during the interval, whether there is adequate variability, and whether the type of incoming data has sufficient signal strength in which to calculate accurate features. Once the data system 210 determines that a sufficient amount of data has been collected to capture typical activity patterns and variability for each participant, the data system 210 may end recording of the accelerometer data. This may result in a significant reduction in data volume, simplifying data analysis and storage. Additionally, the participants may experience less discomfort and inconvenience, as they are not required to wear the accelerometers for unnecessarily extended periods of time.

In some implementations, the data system 210 may be utilized for linguistic content analysis in a cognitive assessment. For example, a research study may investigate the cognitive functioning of elderly participants using speech analysis. Participants may engage in a series of verbal tasks, such as describing a picture or recounting a story (e.g., while their speech is recorded) or responding to a prompt such as, “How are you doing today?” The data system 210 may analyze linguistic content of the recorded speech in real-time (e.g., ten-second intervals). The data system 210 may evaluate parameters, such as vocabulary diversity, speech fluency, and syntactic complexity within short segments of the recordings. If a participant has not provided enough data (e.g., if the features are inconclusive), the participant will be asked to continue speaking or to provide another sample in response to a new prompt to facilitate additional data collection. When the data system 210 determines that the linguistic parameters have reached a stable level, indicating that sufficient cognitive data has been collected, the data system 210 may terminate the recording session. Should the participant give a short, efficient, and quality sample, the participant may be stopped early and told that data collection has ended. This approach may reduce the amount of speech data that needs to be processed and analyzed, while also minimizing the duration of verbal tasks, making the assessment process more efficient and less taxing for the participants.

In some implementations, the data system 210 may be utilized for acoustic feature detection in sleep studies. A sleep study may aim to monitor sleep patterns and disturbances of participants with sleep apnea. The participants may use wearable devices that record various physiological signals, including heart rate, respiratory rate, and acoustic features like snoring and coughing. The data system 210 may utilize multiple modalities from physiological sensors, and may continuously monitor acoustic features of recorded signals. The data system 210 may identify and analyze patterns associated with sleep disturbances, such as frequency and intensity of snoring or coughing episodes, within short time windows. Once the data system 210 confirms that sufficient instances of relevant acoustic features have been captured to provide a comprehensive understanding of the participants'sleep patterns, the data system 210 may terminate further data collection. This may reduce the volume of data that needs to be reviewed and analyzed by researchers, and may decrease the wear time for participants, enhancing their comfort and compliance during the study.

In some implementations, the data system 210 may be utilized to administer an experiment that attempts to detect levels of anxiety from a clinical trial participant's voice when prompted about how their day is going (e.g., via a smartphone). The experiment may test a number (e.g., twenty) people over a number (e.g., three) months. For each sample in which a participant is asked to respond, the data system 210 may collect up to two minutes of voice response, but may record only as long as is necessary. The experiment may want a minimum of fifteen words related to sentiment, and a specific minimum voice quality. The data system 210 may receive information indicating a linguistic target of anxiety, a minimum of thirty seconds of recording time at a minimum voice quality (e.g., 44.1 kilohertz (kHz)), an evaluation window of ten seconds, and a maximum of two minutes of recording time.

In response to the experiment specifications, the data system 210 may monitor each participant's voice recordings every ten seconds, in order to assess a quality of the audio during that time window, may utilize natural language processing (NLP) or large language model (LLM) analytics to determine if the type of words the participant uses are associated with anxiety (e.g., or any emotion at all that can give a positive or negative association with anxiety), and may generate an outcome quantification. Assuming that a minimum of 44.1 kHz has been collected for thirty seconds, while at least fifteen sentiment or emotion-related words are identified in that interval, the voice recording may stop and a message thanking the participant for their contribution may be provided.

In some implementations, a participant may begin to record a response on a smartphone by selecting a button to start recording a response to “How are you doing today?” Once the recording begins, the data system 210 may analyze samples every ten seconds (e.g., a specified time window) in real-time in order and measures the quality and the sentiment content. In a first ten-second sample, if the participant's voice cannot be properly heard, the quality is poor and the sentiment analysis cannot be performed by the data system 210. Thus, the data system 210 may initiate another ten seconds of recording, which appears seamless to the participant in that the recording simply continues. In some implementations, the data system 210 may display a progress bar to the participant, and when recorded audio is bad, the progress bar may not continue and may instruct the participant to move to a place to reduce background noise. Once the quality is improved, the progress bar may move forward. In a next ten seconds, the quality of the recording may be drastically improved but the participant may be talking about traffic rather than sentiment. In such situations, the data system 210 may initiate another ten seconds of recording. In some implementations, when the participant fails to talk about sentiment, the data system 210 may provide a new prompt asking the participant to discuss how they feel. In the following thirty seconds, three assessments, with good quality and sentiment content, may be made by the data system 210. As a result, after fifty seconds, the data system 210 may stop the program since the sample contains sufficient, quality data. In some implementations, the data system 210 may generate and output a report with each observation row summarizing the quality and sentiment analysis as it relates to the target word for each time window. The report may include time stamps and a total time.

In this way, the data system 210 collects data and assesses data collection in real-time. For example, the data system 210 may provide an adaptive and efficient data collection process that minimizes data redundancy, reduces data transmission overhead, and conserves resources. By continuously assessing the data quality and relevance in real-time, the data system 210 may optimize data collection by reducing a volume of data required for analysis, thereby decreasing computational complexity, and facilitating faster data processing and analysis.

Additionally, the data system 210 may utilize feature extraction to extract features from the collected data, and may determine whether data collection requirements are satisfied by the data based on the features. When the data collection requirements are satisfied, the data system 210 may terminate the data collection, conserving resources and reducing stress on users providing the data. When the data collection requirements are not satisfied, the data system 210 may adjust data collection settings and may utilize the adjusted data collection settings to collect additional data. Thus, the data system 210 conserves computing resources, networking resources, and/or the like that would otherwise have been consumed by collecting unnecessary, irrelevant, or low-quality data, generating incorrect conclusions based on the unnecessary, irrelevant, or low-quality data, failing to provide effective treatment based on the incorrect conclusions, unnecessarily collecting data after sufficient data has been captured, and/or the like.

As indicated above, FIGS. 2A-2E are provided as an example. Other examples may differ from what is described with regard to FIGS. 2A-2E. The number and arrangement of devices shown in FIGS. 2A-2E are provided as an example. In practice, there may be additional devices, fewer devices, different devices, or differently arranged devices than those shown in FIGS. 2A-2E. Furthermore, two or more devices shown in FIGS. 2A-2E may be implemented within a single device, or a single device shown in FIGS. 2A-2E may be implemented as multiple, distributed devices. Additionally, or alternatively, a set of devices (e.g., one or more devices) shown in FIGS. 2A-2E may perform one or more functions described as being performed by another set of devices shown in FIGS. 2A-2E.

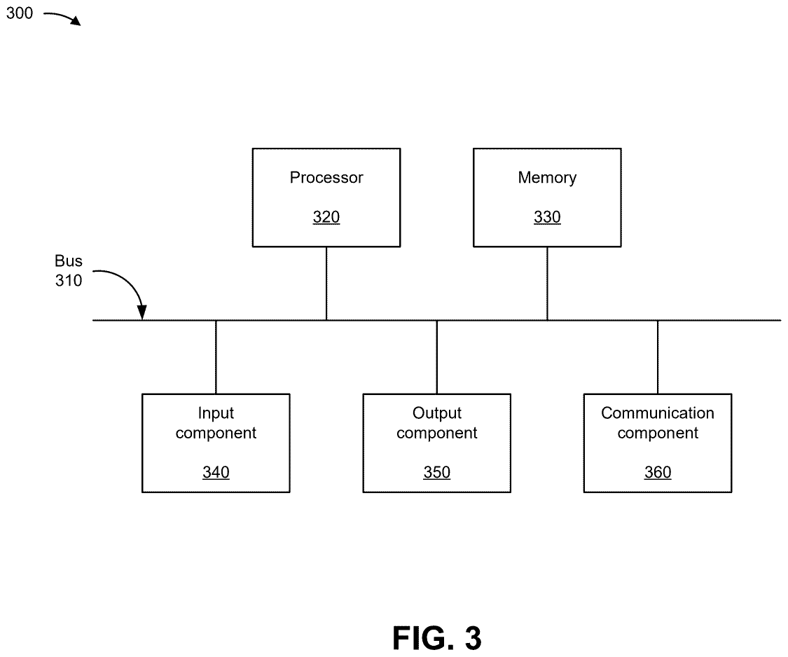

FIG. 3 is a diagram of example components of one or more devices of FIGS. 1 and 2A-2E. The example components may be included in a device 300, which may correspond to computer 102, end user device 106, remote server 108, a device in public cloud 110, a device in private cloud 112, user device 205, and/or data system 210. In some implementations, computer 102, end user device 106, remote server 108, a device in public cloud 110, a device in private cloud 112, user device 205, and/or data system 210 may include one or more devices 300 and/or one or more components of the device 300. As shown in FIG. 3, the device 300 may include a bus 310, a processor 320, a memory 330, an input component 340, an output component 350, and a communication component 360.

The bus 310 includes one or more components that enable wired and/or wireless communication among the components of the device 300. The bus 310 may couple together two or more components of FIG. 3, such as via operative coupling, communicative coupling, electronic coupling, and/or electric coupling. The processor 320 includes a central processing unit (CPU), a graphics processing unit (GPU), a microprocessor, a controller, a microcontroller, a digital signal processor (DSP), a field-programmable gate array (FPGA), an application-specific integrated circuit (ASIC), and/or another type of processing component. The processor 320 is implemented in hardware, firmware, or a combination of hardware and software. In some implementations, the processor 320 includes one or more processors capable of being programmed to perform one or more operations or processes described elsewhere herein.

The memory 330 includes volatile and/or nonvolatile memory. For example, the memory 330 may include RAM, ROM, a hard disk drive, and/or another type of memory (e.g., a flash memory, a magnetic memory, and/or an optical memory). The memory 330 may include internal memory (e.g., RAM, ROM, or a hard disk drive) and/or removable memory (e.g., removable via a universal serial bus connection). The memory 330 may be a non-transitory computer-readable medium. The memory 330 stores information, instructions, and/or software (e.g., one or more software applications) related to the operation of the device 300. In some implementations, the memory 330 includes one or more memories that are coupled to one or more processors (e.g., the processor 320), such as via the bus 310.

The input component 340 enables the device 300 to receive input, such as user input and/or sensed input. For example, the input component 340 may include a touch screen, a keyboard, a keypad, a mouse, a button, a microphone, a switch, a sensor, a global positioning system sensor, an accelerometer, a gyroscope, and/or an actuator. The output component 350 enables the device 300 to provide output, such as via a display, a speaker, and/or a light-emitting diode. The communication component 360 enables the device 300 to communicate with other devices via a wired connection and/or a wireless connection. For example, the communication component 360 may include a receiver, a transmitter, a transceiver, a modem, a network interface card, and/or an antenna.

The device 300 may perform one or more operations or processes described herein. For example, a non-transitory computer-readable medium (e.g., the memory 330) may store a set of instructions (e.g., one or more instructions or code) for execution by the processor 320. The processor 320 may execute the set of instructions to perform one or more operations or processes described herein. In some implementations, execution of the set of instructions, by one or more processors 320, causes the one or more processors 320 and/or the device 300 to perform one or more operations or processes described herein. In some implementations, hardwired circuitry may be used instead of or in combination with the instructions to perform one or more operations or processes described herein. Additionally, or alternatively, the processor 320 may be configured to perform one or more operations or processes described herein. Thus, implementations described herein are not limited to any specific combination of hardware circuitry and software.

The number and arrangement of components shown in FIG. 3 are provided as an example. The device 300 may include additional components, fewer components, different components, or differently arranged components than those shown in FIG. 3. Additionally, or alternatively, a set of components (e.g., one or more components) of the device 300 may perform one or more functions described as being performed by another set of components of the device 300.

FIG. 4 is a flowchart of an example process 400 associated with collecting data and assessing a data collection in real-time. One or more process blocks of FIG. 4 are performed by a computer system (e.g., computer 102 and/or data system 210) and/or by another device or a group of devices separate from or including the computer system. Additionally, or alternatively, one or more process blocks of FIG. 4 may be performed by one or more components of the device 300, such as the processor 320, the memory 330, the input component 340, the output component 350, and/or the communication component 360. Additionally, or alternatively, one or more process blocks of FIG. 4 may be performed by one or more components of computer 102, such as processor set 114, communication fabric 116, volatile memory 118, and/or data collection and assessment code 150.

As shown in FIG. 4, process 400 includes receiving data collection requirements (block 410). For example, the computer system may receive data collection requirements, as described above. In some implementations, receiving the data collection requirements includes providing a user interface for inputting the data collection requirements, and receiving the data collection requirements based on inputs provided via the user interface.

As further shown in FIG. 4, process 400 includes determining data collection settings based on the data collection requirements (block 420). For example, the computer system may determine data collection settings based on the data collection requirements, as described above.

As further shown in FIG. 4, process 400 includes receiving data associated with a user of a user device based on the data collection settings (block 430). For example, the computer system may receive data associated with a user of a user device based on the data collection settings, as described above. In some implementations, receiving the data associated with the user includes receiving sensor data associated with the user in real-time. In some implementations, the data associated with the user includes one or more of a physiological signal associated with the user, a movement pattern of the user, or a speech characteristic of the user.

In some implementations, the data associated with the user includes data associated with one or more of monitoring physical activity of the user, providing a cognitive assessment of the user, or providing a sleep study of the user. In some implementations, receiving the data associated with the user includes receiving first data from a wearable device associated with the user, receiving second data from a smartphone associated with the user, receiving third data from medical equipment associated with the user, and combining the first data, the second data, and the third data to generate the data associated with the user.

As further shown in FIG. 4, process 400 includes performing feature extraction to extract features from the data (block 440). For example, the computer system may perform feature extraction to extract features from the data, as described above.

As further shown in FIG. 4, process 400 includes determining whether the data collection requirements are satisfied by the data based on the features (block 450). For example, the computer system may determine whether the data collection requirements are satisfied by the data based on the features, as described above. In some implementations, determining whether the data collection requirements are satisfied by the data based on the features includes processing the features and the data collection requirements, with one or more of a natural language processing model, a machine learning model, or a large language model, to determine whether the data collection requirements are satisfied.

As further shown in FIG. 4, process 400 includes selectively adjusting the data collection settings based on determining that the data collection requirements are not satisfied and to generate adjusted data collection settings, or terminating collection of the data associated with the user based on determining that the data collection requirements are satisfied (block 460). For example, the computer system may selectively adjust the data collection settings based on determining that the data collection requirements are not satisfied and to generate adjusted data collection settings, or terminate collection of the data associated with the user based on determining that the data collection requirements are satisfied, as described above.

In some implementations, process 400 includes generating instructions based on the data collection settings, and providing the instructions to the user device, wherein receiving the data associated with the user includes receiving the data associated with the user based on the instructions. In some implementations, process 400 includes utilizing the adjusted data collection settings to collect additional data associated with the user. In some implementations, process 400 includes storing the data associated with the user in a data structure based on determining that the data collection requirements are satisfied.

In some implementations, process 400 includes determining a quality of the data associated with the user based on determining that the data collection requirements are satisfied, generating a report identifying the quality of the data associated with the user, and providing the report for display. In some implementations, process 400 includes performing an analysis of the data associated with the user based on determining that the data collection requirements are satisfied.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

The foregoing disclosure provides illustration and description, but is not intended to be exhaustive or to limit the implementations to the precise forms disclosed. Modifications may be made in light of the above disclosure or may be acquired from practice of the implementations. For example, various implementations of this disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

A computer program product embodiment (“CPP embodiment” or “CPP”) is a term used in this disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer-readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, RAM, ROM, erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc), or any suitable combination of the foregoing. A computer-readable storage medium, as that term is used in this disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein.

As used herein, satisfying a threshold may, depending on the context, refer to a value being greater than the threshold, greater than or equal to the threshold, less than the threshold, less than or equal to the threshold, equal to the threshold, not equal to the threshold, or the like.

Although particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

When “a processor” or “one or more processors” (or another device or component, such as “a controller” or “one or more controllers”) is described or claimed (within a single claim or across multiple claims) as performing multiple operations or being configured to perform multiple operations, this language is intended to broadly cover a variety of processor architectures and environments. For example, unless explicitly claimed otherwise (e.g., via the use of “first processor” and “second processor” or other language that differentiates processors in the claims), this language is intended to cover a single processor performing or being configured to perform all of the operations, a group of processors collectively performing or being configured to perform all of the operations, a first processor performing or being configured to perform a first operation and a second processor performing or being configured to perform a second operation, or any combination of processors performing or being configured to perform the operations. For example, when a claim has the form “one or more processors configured to: perform X; perform Y; and perform Z,” that claim should be interpreted to mean “one or more processors configured to perform X; one or more (possibly different) processors configured to perform Y; and one or more (also possibly different) processors configured to perform Z.”

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A method comprising:
receiving data collection requirements;
determining data collection settings based on the data collection requirements;
receiving data associated with a user of a user device based on the data collection settings;
performing feature extraction to extract features from the data;
determining whether the data collection requirements are satisfied by the data based on the features; and
selectively:
adjusting the data collection settings based on determining that the data collection requirements are not satisfied and to generate adjusted data collection settings, or
terminating collection of the data associated with the user based on determining that the data collection requirements are satisfied.

2. The method of claim 1, further comprising:
generating instructions based on the data collection settings; and
providing the instructions to the user device,
wherein receiving the data associated with the user comprises:
receiving the data associated with the user based on the instructions.

3. The method of claim 1, further comprising:
utilizing the adjusted data collection settings to collect additional data associated with the user.

4. The method of claim 1, further comprising:
storing the data associated with the user in a data structure based on determining that the data collection requirements are satisfied.

5. The method of claim 1, wherein receiving the data associated with the user comprises:
receiving sensor data associated with the user in real-time.

6. The method of claim 1, further comprising:
determining a quality of the data associated with the user based on determining that the data collection requirements are satisfied;
generating a report identifying the quality of the data associated with the user; and
providing the report for display.

7. The method of claim 1, wherein the data associated with the user includes one or more of a physiological signal associated with the user, a movement pattern of the user, or a speech characteristic of the user.

8. A computer system, comprising:
a processor set;
one or more computer-readable storage media; and
program instructions stored on the one or more computer-readable storage media to cause the processor set to perform operations comprising:
receiving data collection requirements;
determining data collection settings based on the data collection requirements;
generating instructions based on the data collection settings;
providing the instructions to a user of a user device;
receiving data associated with the user of the user device based on the instructions;
performing feature extraction to extract features from the data;
determining whether the data collection requirements are satisfied by the data based on the features; and
selectively:
adjusting the data collection settings based on determining that the data collection requirements are not satisfied and to generate adjusted data collection settings, or terminating collection of the data associated with the user based on determining that the data collection requirements are satisfied.

9. The computer system of claim 8, wherein determining whether the data collection requirements are satisfied by the data based on the features comprises:
processing the features and the data collection requirements, with one or more of a natural language processing model, a machine learning model, or a large language model, to determine whether the data collection requirements are satisfied.

10. The computer system of claim 8, wherein the operations further comprise:
performing an analysis of the data associated with the user based on determining that the data collection requirements are satisfied.

11. The computer system of claim 8, wherein receiving the data collection requirements comprises:
providing a user interface for inputting the data collection requirements; and
receiving the data collection requirements based on inputs provided via the user interface.

12. The computer system of claim 8, wherein the data associated with the user includes data associated with one or more of monitoring physical activity of the user, providing a cognitive assessment of the user, or providing a sleep study of the user.

13. The computer system of claim 8, wherein receiving the data associated with the user comprises:
receiving first data from a wearable device associated with the user;
receiving second data from a smartphone associated with the user;
receiving third data from medical equipment associated with the user; and
combining the first data, the second data, and the third data to generate the data associated with the user.

14. The computer system of claim 8, wherein determining whether the data collection requirements are satisfied by the data based on the features comprises:
evaluating the features in real-time within predefined time windows to determine whether the data associated with the user satisfies the data collection requirements.

15. A computer program product, comprising:
one or more computer-readable storage media; and
program instructions stored on the one or more computer-readable storage media to perform operations comprising:
receiving data collection requirements;
determining data collection settings based on the data collection requirements;
receiving data associated with a user of a user device based on the data collection settings;
performing feature extraction to extract features from the data;
determining whether the data collection requirements are satisfied by the data based on the features; and
selectively:
adjusting the data collection settings based on determining that the data collection requirements are not satisfied and utilizing the data collection settings, as adjusted, to collect additional data associated with the user, or
terminating collection of the data associated with the user based on determining that the data collection requirements are satisfied.

16. The computer program product of claim 15, wherein the operations further comprise:
storing the data associated with the user in a data structure based on determining that the data collection requirements are satisfied.

17. The computer program product of claim 15, wherein the data associated with the user includes real-time sensor data associated with the user.

18. The computer program product of claim 15, wherein the operations further comprise:
determining a quality of the data associated with the user based on determining that the data collection requirements are satisfied;
generating a report identifying the quality of the data associated with the user; and
providing the report for display.

19. The computer program product of claim 15, wherein the operations further comprise:
performing an analysis of the data associated with the user based on determining that the data collection requirements are satisfied.

20. The computer program product of claim 15, wherein determining whether the data collection requirements are satisfied by the data based on the features comprises:
evaluating the features in real-time within predefined time windows to determine whether the data associated with the user satisfies the data collection requirements.
